(19) United States
(12) Patent Application Publication
(10) Pub. No.: US 2023/0181075 A1
(43) Pub. Date: Jun. 15, 2023

(54) FATIGUE ESTIMATION SYSTEM, FATIGUE ESTIMATION METHOD, AND RECORDING MEDIUM

(71) Applicant: Panasonic Intellectual Property Management Co., Ltd., Osaka (JP)

(72) Inventor: Kazuaki HASHIMOTO, Osaka (JP)

(21) Appl. No.: 17/924,303

(22) PCT Filed: May 19, 2021
(86) PCT No.: PCT/JP2021/018946

§ 371 Date: Nov. 9, 2022

(30) Foreign Application Priority Data
May 28, 2020 (JP) 2020-093032

(51) Int. Cl. A61B 5/16; A61B 5/11; A61B 5/00

(52) U.S. Cl. CPC A61B 5/165; A61B 5/1114; A61B 5/7278; A61B 5/1116; A61B 5/1128; A61B 2503/20

(57) ABSTRACT

A fatigue estimation system includes: an information output device (e.g., an imaging device) that outputs information regarding locations of body parts of a subject; and an estimation device that, based on the information output from the information output device in a predetermined time period, estimates a fatigue level of the subject accumulated in the predetermined time period by counting a specific movement that appears in response to fatigue accumulation, and outputs the estimated fatigue level.

### CROSS-REFERENCE OF RELATED APPLICATIONS

This application is the U.S. National Phase under 35 U.S.C. § 371 of International Patent Application No. PCT/JP2021/018946, filed on May 19, 2021, which in turn claims the benefit of Japanese Patent Application No. 2020-093032, filed on May 28, 2020, the entire disclosures of which Applications are incorporated by reference herein.

### TECHNICAL FIELD

The present disclosure relates to a fatigue estimation system, a fatigue estimation method, and a recording medium for estimating the fatigue level of a subject.

### BACKGROUND ART

In recent years, cases such that the accumulation of fatigue leads to poor health, injuries, accidents, etc. are found here and there. This has brought our attentions to the technique of estimating the level of fatigue to prevent poor health, injuries, accidents, etc. For example, as a fatigue estimation system for estimating a fatigue level that is the level of fatigue, there has been disclosed a fatigue determination device that determines presence or absence of fatigue and the type of the fatigue based on force measurement and bioelectrical impedance analysis (see PTL 1).

### CITATION LIST

### Patent Literature

[PTL 1] Japanese Unexamined Patent Application Publication No. 2017-023311

### SUMMARY OF INVENTION

### Technical Problem

Unfortunately, the fatigue level estimation is not appropriately performed in some cases. In view of this, the present disclosure provides, for instance, a fatigue estimation system that estimates the fatigue level of a subject more appropriately.

### Solution to Problem

A fatigue estimation system according to one aspect of the present disclosure includes: an information output device that outputs information regarding locations of body parts of a subject; and an estimation device that, based on the information output from the information output device in a predetermined time period, estimates a fatigue level of the subject accumulated in the predetermined time period by counting a specific movement that appears in response to fatigue accumulation, and outputs the estimated fatigue level.

A fatigue estimation method according to one aspect of the present disclosure includes: obtaining information regarding locations of body parts of a subject; and based on the information obtained, estimating a fatigue level of the subject accumulated in a predetermined time period by counting a specific movement that appears in response to fatigue accumulation in the predetermined time period.

One aspect of the present disclosure can be implemented as a non-transitory computer-readable recording medium having recorded thereon a program for causing a computer to execute the fatigue estimation method described above.

### Advantageous Effects of Invention

The fatigue estimation system according to one aspect of the present disclosure, for instance, can estimate the fatigue level of a subject more appropriately.

### BRIEF DESCRIPTION OF DRAWINGS

FIG. 1 is a schematic diagram for explaining the overview of a fatigue estimation system according to an embodiment.

FIG. 2 is a block diagram illustrating the functional configuration of the fatigue estimation system according to the embodiment.

FIG. 3 is a diagram for explaining specific movements according to the embodiment.

FIG. 4 is a diagram for explaining personal fatigue information according to the embodiment.

FIG. 5 is a diagram for explaining a method for constructing personal fatigue information according to the embodiment.

FIG. 6 is a diagram for explaining fatigue level estimation according to the embodiment.

FIG. 7 is a diagram for explaining a blank period according to the embodiment.

FIG. 8 is a diagram for explaining fatigue level correction according to the embodiment.

FIG. 9 is a first diagram illustrating information to be output from the fatigue estimation system according to the embodiment.

FIG. 10 is a second diagram illustrating information to be output from the fatigue estimation system according to the embodiment.

FIG. 11 is a flowchart illustrating an operation of the fatigue estimation system according to the embodiment.

### DESCRIPTION OF EMBODIMENTS

Hereinafter, embodiments of the present disclosure will be described with reference to the drawings. Each of the embodiments described below illustrates a generic or specific example. Moreover, numerical values, shapes, materials, elements, arrangement and connection of the elements, steps, an order of steps, etc. described in the following embodiments are mere examples and are not intended to limit the present disclosure. Among elements described in the following embodiments, those not recited in any one of the independent claims are described as optional elements.

The drawings are schematic and are not necessarily accurate illustrations. Elements having substantially same configurations are assigned with like reference signs in the drawings, and duplicate description may be omitted or simplified.

### Embodiment

### [System Configuration]

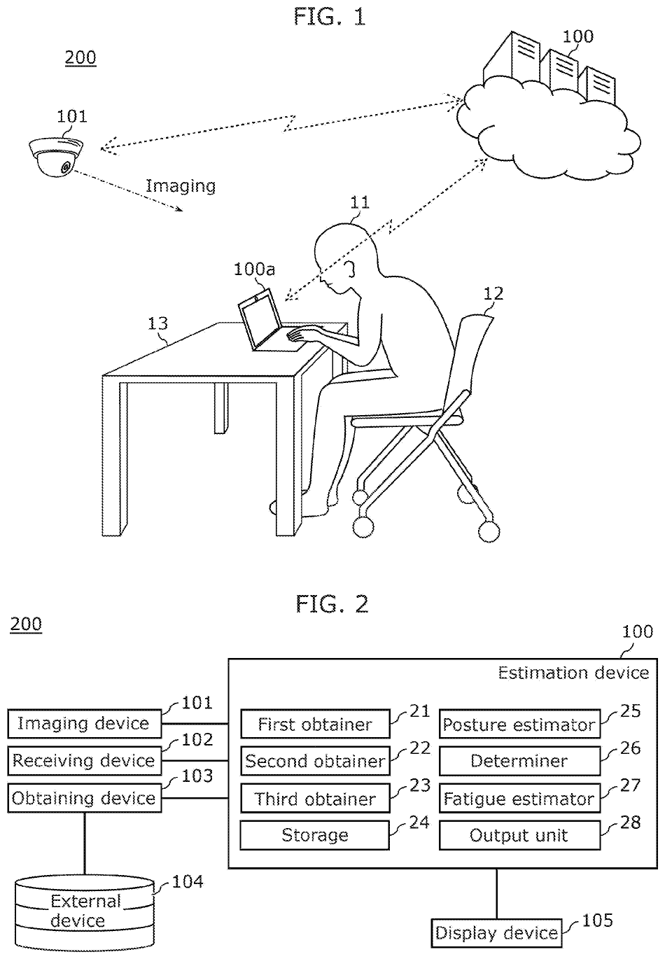

First, the overall configuration of a fatigue estimation system according to an embodiment will be described with reference to FIG. 1 and FIG. 2. FIG. 1 is a schematic diagram for explaining the overview of the fatigue estimation system according to the embodiment. FIG. 1 illustrates how the fatigue level of subject 11 is estimated using fatigue estimation system 200. In the scene illustrated in FIG. 1, subject 11 sits on chair 12 and operates computer 100a placed on desk 13.

In the present embodiment, fatigue estimation system 200 estimates the fatigue level of subject 11 based on images of subject 11 captured by imaging device 101. The images captured by imaging device 101 are transmitted to estimation device 100 via a network such as the Internet. Estimation device 100 is, for example, a computing device mounted on a server device such as a cloud server, and estimates, based on images, the fatigue level of subject 11 included in each of the images. The result of the estimation is, for example, transmitted to computer 100a operated by subject 11 via a network, and displayed on the screen of computer 100a or stored in a storage device (such as storage 24 which is to be described later).

In this case, subject 11 can check, while working using computer 100a, an estimation result displayed on the same computer 100a. The present embodiment illustrates an example in which estimation device 100 is implemented by a server device, as described above, but the configuration of fatigue estimation system 200 is not limited to such an example. For example, estimation device 100 may be built into computer 100a. In other words, computer 100a is an estimation device in another embodiment.

When using computer 100a as an estimation device, it is possible to implement fatigue estimation system 200 with a simple configuration including imaging device 101 and computer 100a since there is no need for fatigue estimation system 200 to include a network and a server device. Computer 100a may be provided with a camera at a location that enables capturing images of subject 11, and by using the camera as imaging device 101 described above, it is also possible to implement fatigue estimation system 200 with computer 100a alone.

In the present disclosure, when estimation device 100 estimates the fatigue level of subject 11 from the posture of subject 11, it is possible to estimate, through simple computing, the fatigue level of subject 11 accumulated in a predetermined time period, by counting, in the predetermined time period, a specific movement that appears in response to the fatigue accumulation of subject 11. The predetermined time period is a time period set by a user of fatigue estimation system 200, such as a manager managing subject 11 or the fatigue level of subject 11, and any period of one hour, eight hours, one day, three days, one week, one month, etc. may be set. The present embodiment describes fatigue estimation system 200 that sets one day for the predetermined time period and estimates a fatigue level indicating the level of fatigue accumulated in subject 11 in a day.

Since a relationship between the count of such a specific movement and a fatigue level to be accumulated may vary from subject 11 to subject 11, it is possible to obtain a fatigue level estimation result appropriate for subject 11 by using personal fatigue information constructed in advance for subject 11. Accordingly, it is possible to estimate the fatigue level of subject 11 through simple computing, and also achieve fatigue level estimation adapted to each subject 11.

FIG. 2 is a block diagram illustrating the functional configuration of the fatigue estimation system according to the embodiment. As illustrated in FIG. 2, fatigue estimation system 200 according to the present embodiment includes estimation device 100, imaging device 101, receiving device 102, obtaining device 103, external device 104, and display device 105.

As described above, estimation device 100 is a processing device that estimates a fatigue level indicating the level of fatigue accumulated in subject 11, and is implemented by being mounted on a server device. Estimation device 100 includes first obtainer 21, second obtainer 22, third obtainer 23, storage 24, posture estimator 25, determiner 26, fatigue estimator 27, and output unit 28.

First obtainer 21 is a communication module that obtains images in each of which subject 11 is captured. For example, first obtainer 21 obtains images captured by imaging device 101 by communicating with imaging device 101 via a network.

Imaging device 101 is a device that outputs images each including subject 11 by capturing the images, and is implemented by a camera installed in a facility, such as a security camera, or a camera built into, for instance, computer 100a or a mobile device, or a dedicated camera for use in fatigue estimation system 200. Images output by imaging device 101 and obtained by first obtainer 21 are so-called a video sequentially captured in time series. First obtainer 21 obtains such a video in parallel to image capturing performed by imaging device 101. First obtainer 21 outputs the obtained images to posture estimator 25.

Posture estimator 25 is a processing unit that estimates the posture of subject 11 based on images output from first obtainer 21. Posture estimator 25 is implemented by a predetermined program being executed by, for instance, a processor and memory. As described above, since the images are a video composed by a sequence of frame images in time series, posture estimator 25 estimates the posture of subject 11 in each of the frame images composing the video. Accordingly, the estimated postures of subject 11 are output from posture estimator 25 through the entire time period in which fatigue level estimation is performed. Note, however, that when subject 11 is outside the field of view of imaging device 101, posture estimator 25 may stop estimating the posture of subject 11.

Posture estimator 25 localizes the joint positions of subject 11 in an image by performing image processing using a predetermined program. Posture estimator 25 outputs, as the result of the posture estimation, a joint position model expressed by connecting two joints by a bone having a predetermined length based on the relative positions of the joints. A joint position model may be read as a skeletal position model since the relative positions of joints are in one-to-one correspondence with the relative positions of bones connecting the joints. Estimation device 100 estimates the fatigue level of subject 11 by counting a specific movement that appears in response to fatigue accumulation in subject 11.

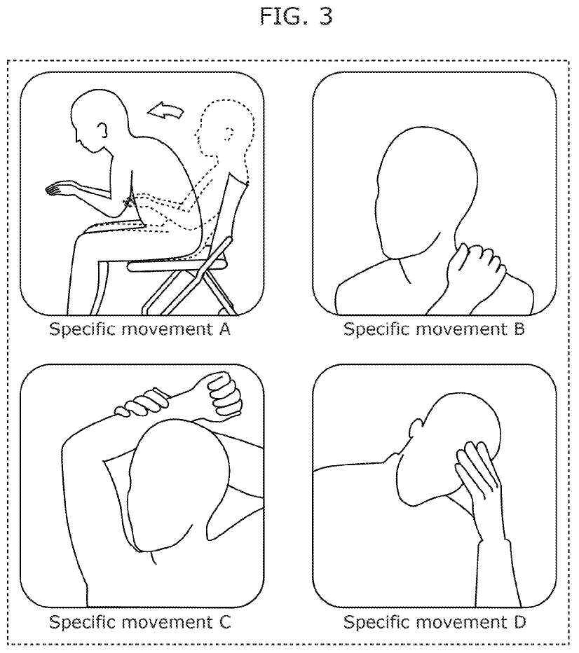

Personal fatigue information is stored in storage 24 as information related to a specific movement. Storage 24 is a storage device implemented by, for instance, a semiconductor memory, a magnetic storage medium, or an optical storage medium. Storage 24 stores various types of information that are used in estimation device 100 and include personal fatigue information. Each of processing units in estimation device 100 reads necessary information from storage 24 to use the information, and if necessary, newly writes information generated or the like by the processing unit into storage 24. Specific movements and personal fatigue information will be described later with reference to FIG. 3 through FIG. 5.

The counting of a specific movement, which is based on the posture of subject 11 estimated by posture estimator 25, is performed based on a determination of whether the movement of subject 11 due to a change in the estimated posture of subject 11 matches the specific movement. The determination is performed by determiner 26. Determiner 26 is a processing unit having functions as described above, and is implemented by a predetermined program being executed by, for instance, a processor and memory. As described above, determiner 26 determines whether a movement based on the estimated posture of subject 11 matches a specific movement, to determine whether the specific movement is made. When determining that the movement of subject 11 matches the specific movement, determiner 26 increments the count of the specific movement by 1.

Fatigue estimator 27 is a processing unit that estimates the fatigue level of subject 11 based on the count of a specific movement. Fatigue estimator 27 is implemented by a predetermined program being executed by, for instance, a processor and memory. The detailed operation of fatigue estimator 27 will be described later.

When estimating the fatigue level of subject 11, fatigue estimator 27 corrects a fatigue level calculated based on the count of a specific movement, to perform more accurate fatigue level estimation. In addition to fatigue estimator 27, second obtainer 22 and third obtainer 23 are involved in the correction of the fatigue level. Second obtainer 22 is a communication module that obtains a feeling of fatigue that is input by subject 11 and is based on the subjective view of subject 11. Second obtainer 22 obtains the feeling of fatigue input by subject 11 by, for example, communicating with receiving device 102 via a network.

Receiving device 102 is a device that receives input from subject 11, and is implemented by a device such as an interface device. Fatigue estimation system 200 allows subject 11 to input the degree of fatigue he/she subjectively feels, and corrects the calculated fatigue level using the feeling of fatigue that has been input. The correction using the feeling of fatigue will be described later. The feeling of fatigue includes information comparable with and equivalent to the fatigue level of subject 11.

Third obtainer 23 is a communication module that obtains personal information of subject 11. Third obtainer 23 obtains, for example, a medical examination result including personal information of subject 11 by communicating with obtaining device 103 via a network. Obtaining device 103 obtains, for example, a medical examination result including personal information of subject 11 by communicating, via a network, with external device 104 in which the medical examination result is stored, or the like. External device 104 is, for example, a server in a facility such as a hospital that provides medical examination, a server in an agency that intermediates for the provision of medical examination, or a company server in which the medical examination results of employees including subject 11 are stored. Third obtainer 23 may merely obtain personal information that has been input by subject 11 himself/herself via receiving device 102 or the like.

The personal information of subject 11 includes at least one of age, sex, height, weight, a muscle mass, a stress level, a proportion of fat in a body, or proficiency in performing exercise. The age of subject 11 may be a specific numerical value, or an age range sectioned by ten years as in expressions such as teenage, twenties, and thirties, or an age range defined by two sections with a predetermined age as a border as in an expression such as below 59 or over 60, or any other age range.

The sex of subject 11 is an appropriate one selected from two options of male and female. Numerical values are obtained for the height and weight of subject 11. The compositional ratio of muscles of subject 11 which is measured using, for instance, a body composition analyzer is obtained as the muscle mass of subject 11. The stress level is information determined through selection made by subject 11 himself/herself from among, for instance, high, intermediate, and low as the subjective degree of stress subject 11 feels.

The proficiency of subject 11 in performing exercise may be quantified by scores attained when subject 11 performs exercise in a predetermined program, or by conditions in which subject 11 performs exercise that subject 11 usually takes. In the former case, the proficiency is quantified by, for example, a time required for ten times of back extension, a time required for running 50 meters, or a flying distance achieved in making a long throw. In the latter case, the proficiency is quantified by, for example, how many days subject 11 performs exercise during a week or how many hours subject 11 performs exercise. Since personal information is used with the view to improve the accuracy of a fatigue level to be estimated, fatigue estimation system 200 may be implemented without third obtainer 23, obtaining device 103, and external device 104 when a satisfactory degree of accuracy is ensured.

Fatigue estimator 27 corrects, based on obtained personal information, a fatigue level calculated based on the result of counting a specific movement, to estimate a fatigue level to be finally output from estimation device 100. In the correction of the fatigue level using personal information, the fatigue level may be, for example, decreased as the age of subject 11 gets closer to the peak age of muscle development and increased as the age gets away from the peak age. Such a peak age may be determined based on the sex of subject 11. The fatigue level may be decreased when the sex of subject 11 is male and increased when the sex is female. Alternatively, the fatigue level may be decreased as the height and weight of subject 11 indicate smaller values and increased as the height and weight indicate larger values.

The fatigue level may be decreased as the muscle mass of subject 11 has a higher compositional rate and increased as the muscle mass has a lower compositional rate. Alternatively, the fatigue level may be decreased as the stress level of subject 11 gets lower and increased as the stress level gets higher. Alternatively, the fatigue level may be increased as the proportion of fat in the body of subject 11 gets higher and decreased as the proportion gets lower. Furthermore, the fatigue level may be decreased as the proficiency of subject 11 in performing exercise gets higher and increased as the proficiency gets lower.

As described above, fatigue estimator 27 further corrects a fatigue level estimated based on the count of a specific movement, to perform more accurate fatigue level estimation for each subject 11. Fatigue estimator 27 outputs a fatigue level obtained through estimation to output unit 28.

Output unit 28 is a processing unit that outputs presentation information for presenting an estimation result including an estimated fatigue level to subject 11. Output unit 28 is implemented by a predetermined program being executed by a processor and memory, for instance. Output unit 28 generates image data which is presentation information including the fatigue level of subject 11 estimated by fatigue estimator 27 and other information, and transmits the image data to display device 105 via a network. Output unit 28 may generate audio data as presentation information, in which case output unit 28 transmits the audio data to a sound emission device (not shown in FIG. 2).

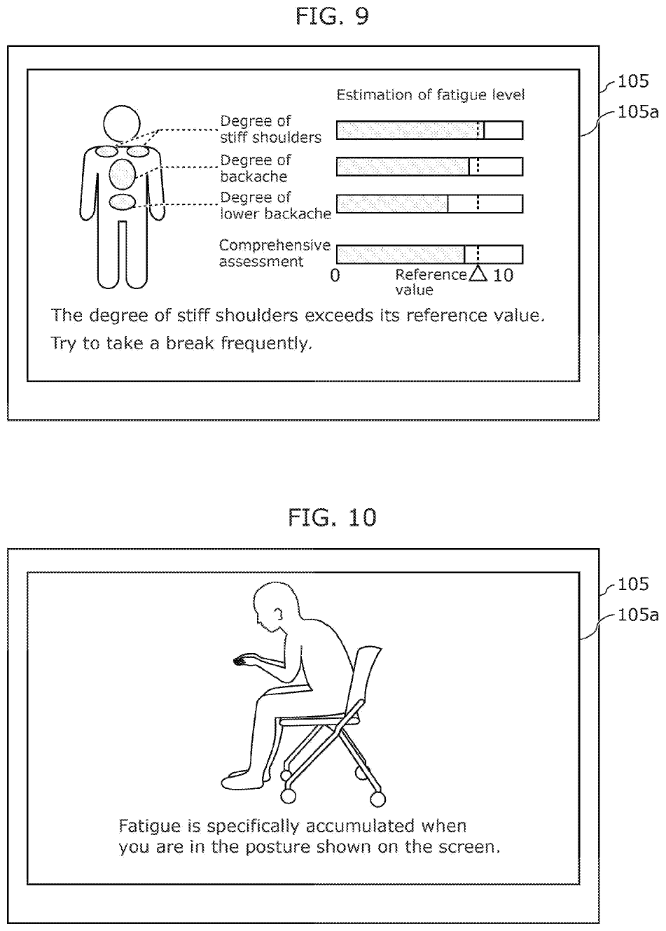

Display device 105 is a device that displays received image data. Display device 105 is a display having display module 105a (see FIG. 9 which is described later) such as a liquid crystal panel, and drives display module 105a to display image data received from output unit 28.

Hereinafter, specific movements and personal fatigue information will be described with reference to FIG. 3 through FIG. 5. FIG. 3 is a diagram for explaining specific movements according to the embodiment. FIG. 3 schematically illustrates persons each making a specific movement. Although FIG. 3 illustrates four types of examples of the specific movement, the number of types of the specific movement used in the present embodiment is not specifically limited.

As described above, a specific movement is a movement a person may make when fatigue is accumulated. For example, one example of the specific movement, which is illustrated as specific movement A in FIG. 3, is a movement in which a person changes from a backward leaning posture to a forward leaning posture. When a person is engaged in some kind of work in a backward leaning posture, fatigue accumulates in the lower back. The person therefore makes specific movement A of changing the posture from the backward leaning posture to the forward leaning posture in order to reduce the accumulated fatigue.

For example, one example of the specific movement, which is illustrated as specific movement B in FIG. 3, is a movement in which a person rubs his/her shoulder. When a person continuously uses the shoulder muscles by, for instance, continuing working with the arms raised, the muscles become hard and the person has so-called stiff shoulders. The person makes specific movement B of massaging the shoulder muscles in order to relax the hardened muscles.

For example, one example of the specific movement, which is illustrated as specific movement C in FIG. 3, is a movement in which a person stretches the back muscles by upwardly stretching his/her arms. When a person continues working in a sitting or standing posture without moving the body so much, the back muscles contract. The person makes specific movement C of upwardly stretching his/her arms that move together with the back muscles, to stretch the contracted back muscles.

For example, one example of the specific movement, which is illustrated as specific movement D in FIG. 3, is a movement in which a person holds his/her forehead. When the blood flow of a person becomes deteriorated due to the state of, for instance, stiff shoulders as in the case described above, the symptoms of a headache may occur and the person makes specific movement D of touching the aching head in such a manner to hold his/her forehead. These specific movements are each composed of information comparable with and equivalent to a joint position model for comparison with the posture of subject 11 estimated by posture estimator 25, as described above. Since each of the specific movements is defined by a plurality of postures continuously changing, the estimated posture is compared with both a plurality of joint position models and an order in which the joint position models change. In addition, since it is rare that a specific movement and the estimated posture match each other perfectly, an allowed range is provided, both in a time domain and a spatial domain, for a posture determined as matching the specific movement.

A specific movement corresponds to a movement that a person in general makes when fatigue (or a load imposed on a joint or a muscle, which is equivalent to fatigue) is accumulated in the person. The specific movement is not limited to the four types of movements described above, and any movement that appears when fatigue is accumulated may be applied. The specific movement may also include a movement unique to subject 11. In other words, a movement, which appears very frequently when fatigue is accumulated in subject 11 although the movement rarely appears when fatigue is accumulated in a person in general, may be included as a specific movement. In the present embodiment, by thus properly defining a specific movement included in personal fatigue information, it is also possible to specialize, for subject 11, the estimation of the fatigue level of subject 11 performed by estimation device 100.

Hereinafter, an example of estimating the fatigue level of subject 11 using the four types of specific movements described above will be illustrated. Since the specific movements used herein are each a movement that generally appears at the time of fatigue, estimation device 100 applicable to fatigue estimation for everyone in general is achieved. In the following description, specific movements A through D may be used without any specific description thereof, in which case the details of each of the specific movements are omitted by referring to the description of FIG. 3 provided above.

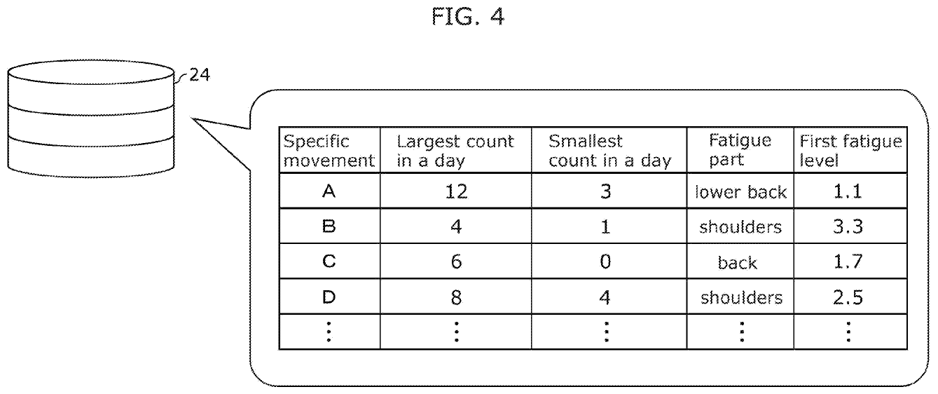

FIG. 4 is a diagram for explaining personal fatigue information according to the embodiment. FIG. 4 illustrates personal fatigue information stored in storage 24. As illustrated in the figure, in the personal fatigue information, each of the specific movements is associated with information corresponding to subject 11 who is a predetermined individual. In other words, in the case where the fatigue levels of a plurality of subjects 11 are estimated using fatigue estimation system 200, a plurality of sets of personal fatigue information are prepared in one-to-one correspondence with the plurality of subjects 11.

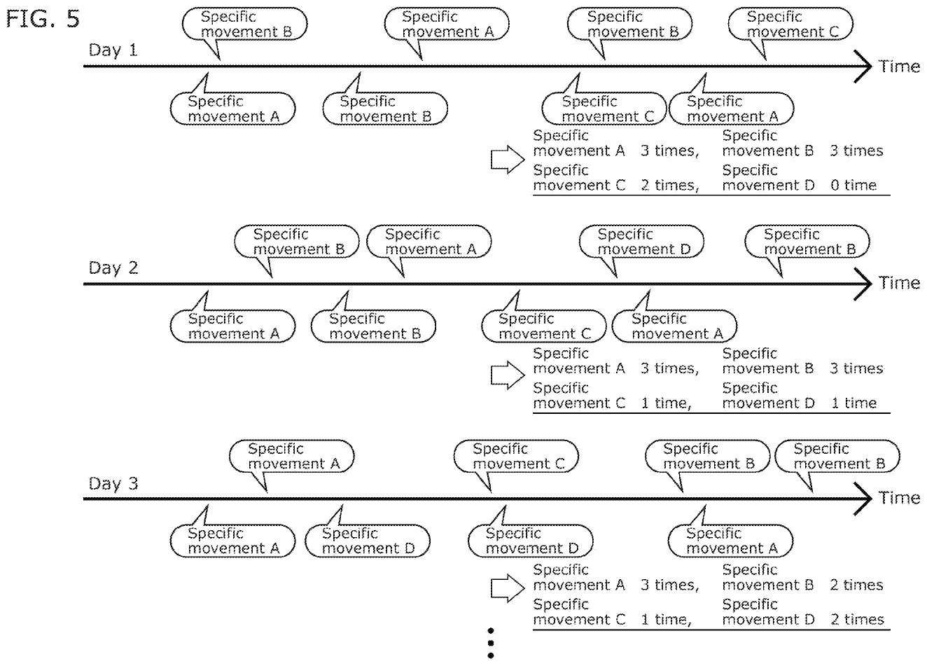

As illustrated in FIG. 4, each of the specific movements is associated with the largest number of counts in a day that is a predetermined time period (the largest count in a day) and the smallest number of counts in a day (the smallest count in a day). For example, the largest count in a day associated with specific movement A is 12 times and the smallest count in a day associated with specific movement A is 3 times. A method of determining the largest count in a day and the smallest count in a day will be described. FIG. 5 is a diagram for explaining a method for constructing personal fatigue information according to the embodiment. FIG. 5 illustrates a method of determining the largest count in a day and the smallest count in a day which are used for constructing personal fatigue information.

When one day is set as a predetermined time period, as is the case of the present embodiment, counting a specific movement made by subject 11 during a day is performed. For example, specific movement A is counted 3 times, specific movement B is counted 2 times, specific movement C is counted 2 times, and specific movement D is counted 0 time on Day 1. Specific movement A is counted 3 times, specific movement B is counted 3 times, specific movement C is counted 1 time, and specific movement D is counted 1 time on Day 2. Specific movement A is counted 3 times, specific movement B is counted 2 times, specific movement C is counted 1 time, and specific movement D is counted 2 times on Day 3.

By thus counting each of the specific movements repeatedly over a plurality of days corresponding to a plurality of predetermined time periods, it is possible to obtain the count of the specific movement on the day the specific movement is made the most frequently and the count of the specific movement on the day the specific movement is made the least frequently. The obtained count of the specific movement on the day the specific movement is made the most frequently and the obtained count of the specific movement on the day the specific movement is made the least frequently are determined as the largest count in a day and the smallest count in a day, respectively.

The accuracy and precision of the largest count in a day and the smallest count in a day of a specific movement vary depending on the number of predetermined time periods (i.e., the number of days) over which the counting of the specific movement is performed. A user of fatigue estimation system 200 is therefore recommended to count each of the specific movements, as described above, until the largest count in a day and the smallest count in a day are obtained with desired accuracy and precision, and construct personal fatigue information.

Referring back to FIG. 4, a first fatigue level to be accumulated every time subject 11 makes specific movement A is calculated based on the largest count in a day and the smallest count in a day which are determined as described above. More specifically, the first fatigue level of each of the specific movements is calculated by an expression that is 10/{(the largest count in a day)−(the smallest count in a day)} using the largest count in a day and the smallest count in a day which are associated with the specific movement. A first fatigue level is a value indicating the amount of a fatigue level to be accumulated every time a specific movement is counted, and is a value uniquely determined by the largest count in a day and the smallest count in a day of the specific movement.

As illustrated in FIG. 4, personal fatigue information includes a first fatigue level and information (the largest count in a day and the smallest count in a day, here) related to the first fatigue level for determining the first fatigue level. Since a first fatigue level is a value uniquely determined based on information related to the first fatigue level, if personal fatigue information includes information related to a first fatigue level, a first fatigue level per se does not need to be included in the personal fatigue information. The expression described above is used in the present embodiment where a first fatigue level is scored by 10 points. When scoring a first fatigue level by other value, it is recommended to change the numerical value of 10 in the numerator in the expression.

In FIG. 4, the first fatigue level of specific movement A is derived by 10/(12−3)≈1.1, the first fatigue level of specific movement B is derived by 10/(4−1)≈3.3, the first fatigue level of specific movement C is derived by 10/(6−0)≈1.7, and the first fatigue level of specific movement D is derived by 10/(8−4)=2.5. These first fatigue levels for another subject 11 are, for example, as follows: the first fatigue level of specific movement A is 2.4; the first fatigue level of specific movement B is 1.0; the first fatigue level of specific movement C is 5.0; and the first fatigue level of specific movement D is 3.3 (none of which is shown in the figure). A fatigue level accumulated until a specific movement is made thus varies from subject 11 to subject 11. In the present embodiment, personal fatigue information is constructed for each subject 11 to enable fatigue level estimation in which personal habits in specific movements are reflected.

As illustrated in FIG. 4, personal fatigue information according to the present embodiment includes fatigue part information that associates a specific movement with a fatigue part which is a body part of subject 11 for which a first fatigue level that is set for each specific movement is accumulated. For example, the fatigue part information associates specific movement A with a lower back which is a fatigue part corresponding to specific movement A. In other words, when subject 11 makes specific movement A, the first fatigue level of 1.1 is accumulated for the lower back of subject 11. Similarly, when subject 11 makes specific movement B, the first fatigue level of 3.3 is accumulated for the shoulders of subject 11, when subject 11 makes specific movement C, the first fatigue level of 1.7 is accumulated for the back of subject 11, and when subject 11 makes specific movement D, the first fatigue level of 2.5 is accumulated for the shoulders of subject 11.

Specific movement B and specific movement D are associated with shoulders which are same as a fatigue part. In this case, the fatigue level of the shoulders may be calculated by averaging fatigue levels finally obtained for specific movements B and D. Alternatively, the fatigue level may be calculated by mingling the fatigue levels of specific movements B and D at a ratio according to weighting coefficients previously set for specific movements B and D. The weighting coefficients are determined based on the frequency and count of each specific movement which are used for constructing personal fatigue information.

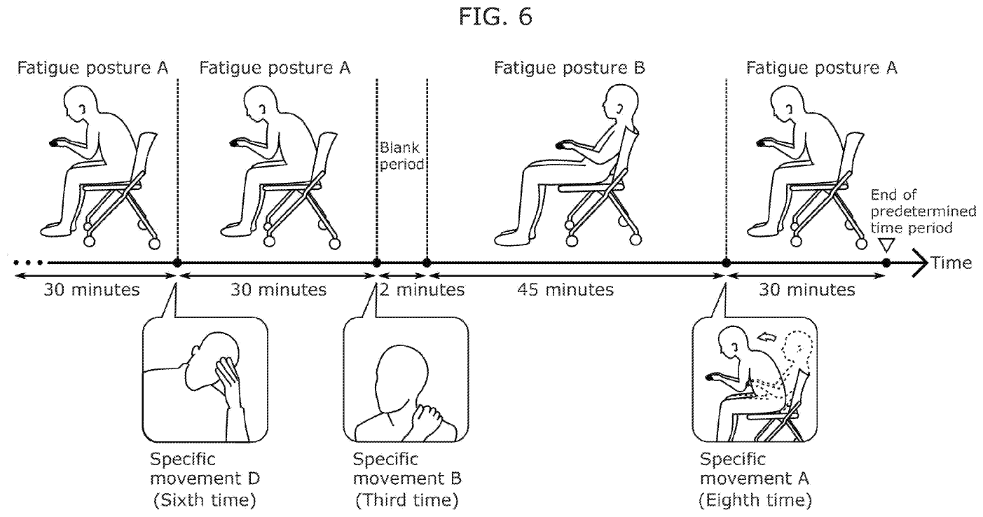

Next, estimation of the fatigue level of subject 11 and others will be described with reference to FIG. 6 through FIG. 8. FIG. 6 is a diagram for explaining fatigue level estimation according to the embodiment. FIG. 6 illustrates postures of subject 11 and timings at each of which a specific movement is made during a time period from the middle to the end of a predetermined time period along a time series. Specifically, after continuing working in a forward leaning posture (hereinafter referred to as fatigue posture A) for 30 minutes, subject 11 makes specific movement D and returns to fatigue posture A in the time period illustrated in FIG. 6. After continuing working in fatigue posture A for another 30 minutes, subject 11 makes specific movement B and a blank period of 2 minutes is generated due to subject 11 being out of the field of view of imaging device 101. After continuing working in a backward leaning posture (hereinafter referred to as fatigue posture B) for 45 minutes, subject 11 makes specific movement A and returns to fatigue posture A. After subject 11 continues working in fatigue posture A for 30 minutes, the predetermined time period ends. The description will continue under the assumption that specific movement C is made 5 times.

In this case, determiner 26 counts 8 times for specific movement A, 3 times for specific movement B, 5 times for specific movement C, and 6 times for specific movement D. When the fatigue level of subject 11 is calculated based on the count of a specific movement, standardization is performed where a fatigue level corresponding to the smallest count in a day is defined as fatigue level 0. Accordingly, fatigue estimator 27 multiplies, by the first fatigue level of the specific movement, a difference obtained by subtracting the smallest count in a day of the specific movement from the count of the specific movement. For example, fatigue estimator 27 calculates 1.1×(8−3)=5.5 since specific movement A is made 8 times, and calculates that the fatigue level of 5.5 is accumulated for the lower back of subject 11 based on the count of the specific movement.

Similarly, fatigue estimator 27 calculates that the fatigue level of 6.6 is accumulated for the shoulders based on the count of specific movement B, the fatigue level of 8.5 is accumulated for the back based on the count of specific movement C, and the fatigue level of 5.0 is accumulated for the shoulders based on the count of specific movement D. The fatigue level calculated based on the count of specific movement B and the fatigue level calculated based on the count of specific movement D are both accumulated for the shoulders that are the same fatigue part, and fatigue estimator 27 calculates the average value of these fatigue levels as the fatigue level of the shoulders. Specifically, the fatigue level of the shoulders is 5.8 in this case.

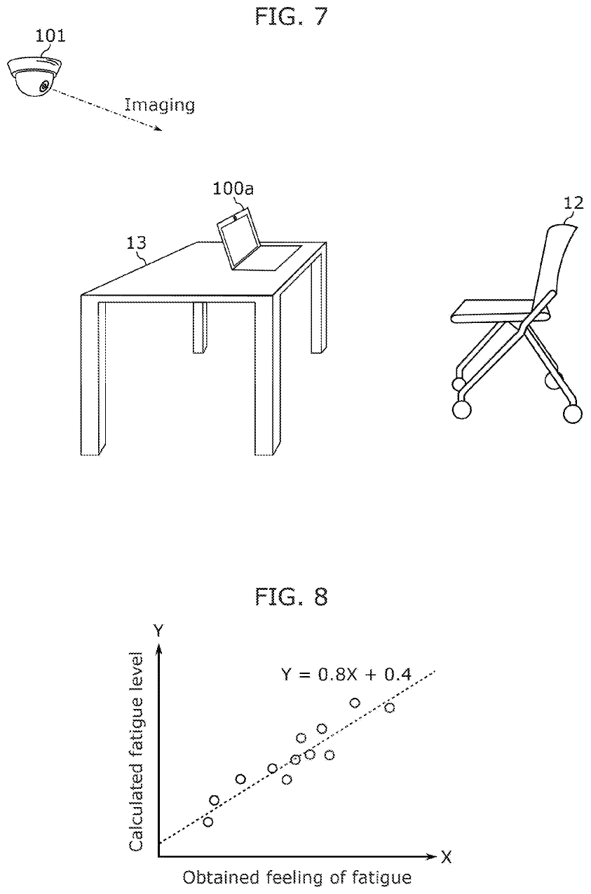

Next, correction of the fatigue level of subject 11 in a blank period will be described with reference to FIG. 7. FIG. 7 is a diagram for explaining a blank period according to the embodiment. As illustrated in FIG. 7, when subject 11 is out of the field of view of imaging device 101, a blank period in which fatigue level estimation based on images cannot be performed is generated. In view of this, in the present embodiment, when such a blank period is generated, fatigue estimator 27 accumulates a preset supplementary fatigue level in accordance with the length of the blank period and adds, to a previously calculated fatigue level, a numerical value obtained as a result of the accumulation of the preset supplementary fatigue level.

The example in FIG. 6, for example, is the case where subject 11 takes a two-minute break, and fatigue estimator 27 accumulates a supplementary fatigue level of −0.05 per minute, for example. Accordingly, the fatigue level of subject 11 is partly reduced and it is calculated that the fatigue level of 5.7 is accumulated for the shoulders, the fatigue level of 8.4 is accumulated for the back, and the fatigue level of 5.4 is accumulated for the lower back. In the case where subject 11 carries out a different work outside the field of view of imaging device 101 in a blank period, a supplementary fatigue level is accumulated in accordance with the work and the fatigue level of subject 11 increases. Regarding the action of subject 11 in a blank period, the schedule of subject 11 may be obtained from an external schedule management server or the like (not shown in the diagrams) so that the action is automatically determined, or subject 11 himself/herself may input his/her schedule to fatigue estimation system 200.

Next, in the present embodiment, fatigue estimator 27 performs correction for taking into account a fatigue level to be accumulated due to fatigue posture A after the eighth specific movement A in FIG. 6. Specifically, fatigue estimator 27 stores, in storage 24 or the like, the postures of subject 11 in a time period from a predetermined timing until subject 11 makes a specific movement in the predetermined time period, together with a fatigue level to be accumulated per unit time. Using the stored information, fatigue estimator 27 estimates the fatigue level of subject 11 after a specific movement is last made. The predetermined timing is, for instance, when a predetermined time period is started, immediately after a specific movement is made, or immediately after a blank period. Stated differently, the duration of a single fatigue posture is sandwiched between a predetermined timing and the timing of a specific movement. Fatigue estimator 27 calculates a fatigue level to be accumulated per unit time due to a fatigue posture thus sandwiched between a predetermined timing and the timing of a specific movement.

In the example in FIG. 6, for example, after continuing, for 30 minutes, fatigue posture A that is before the sixth specific movement D, subject 11 makes specific movement D. It is considered that fatigue causing subject 11 to make specific movement D is brought by fatigue posture A of 30 minutes. Accordingly, fatigue estimator 27 calculates that the fatigue level (i.e., a second fatigue level) of fatigue posture A to be accumulated per minute is 0.08 as a result of dividing the first fatigue level 2.5 of specific movement D by 30 minutes. Since the shoulders are set for the fatigue part of specific movement D, the fatigue level of 0.08 accumulated per minute for the shoulders is stored also for fatigue posture A in storage 24.

Similarly, fatigue estimator 27 calculates the second fatigue level of the shoulders as 3.3/30 minutes=0.11 for fatigue posture A that is after the sixth specific movement D and before the third specific movement B in FIG. 6, and stores the calculated second fatigue level in storage 24. In each of the two calculation examples described above, the second fatigue level to be accumulated for the shoulders due to fatigue posture A is calculated, but there is a difference between the two calculated values. Accordingly, fatigue estimator 27 calculates the average value of these values to determine that the fatigue level of 0.10 is accumulated per minute for the shoulders due to fatigue posture A, and updates the information stored in storage 24. Fatigue estimator 27 calculates a second fatigue level for other fatigue posture in the same manner, and stores the second fatigue level in storage 24.

After that, fatigue estimator 27 refers to second fatigue levels stored in storage 24 for a time period in which fatigue level estimation using specific movements cannot be carried out, as is the case of fatigue posture A after the eighth specific movement A in FIG. 6, to estimate the fatigue level of subject 11 in the predetermined time period including the time period. In the example in FIG. 6, for example, subject 11 takes fatigue posture A in a time period in which fatigue level estimation cannot be performed. Accordingly, assuming that the fatigue level of 0.10 is accumulated per minute for the shoulders, 30 minutes×0.10=3.0 is derived. Consequently, fatigue estimator 27 estimates that, in addition to previously calculated fatigue levels, the fatigue level of 8.7 is accumulated for the shoulders, the fatigue level of 8.4 is accumulated for the back, and the fatigue level of 5.4 is accumulated for the lower back.

Since the estimation result described above is obtained as a result of estimation based merely on captured images, the estimation result may not match a feeling of fatigue subject 11 is actually having. When a gap between an estimation result and a feeling of fatigue based on the subjective view of subject 11 occurs, subject 11 may experience a feeling of strangeness. In the present embodiment, fatigue estimator 27 performs correction based on a feeling of fatigue that is based on the subjective view of subject 11 so that the fatigue level of subject 11 is estimated with consideration given to the feeling of fatigue that is based on the subjective view of subject 11. Specifically, fatigue estimator 27 receives, as input, information related to a feeling of fatigue from subject 11, and corrects the fatigue level based on the received information.

For example, fatigue estimation system 200 displays a question such as “How tired do you think you are?” by output unit 28 and display device 105 after the end of a predetermined time period, and obtains the feeling of fatigue of subject 11 as a response to the question. Input from subject 11 is received via receiving device 102 and obtained by second obtainer 22. The obtained feeling of fatigue includes a feeling of fatigue for the shoulders, a feeling of fatigue for the back, and a feeling of fatigue for the lower back. Fatigue estimator 27 outputs, as the estimated value of the fatigue level of each of the body parts, the average value of the obtained feeling of fatigue and the calculated fatigue level of the body part.

It is assumed herein that the feeling of fatigue 7.0 for the shoulders, the feeling of fatigue 7.0 for the back, and the feeling of fatigue 6.0 for the lower back are input by subject 11, for example. Fatigue estimator 27 calculates the average value of the obtained feeling of fatigue and the calculated fatigue level. Fatigue estimator 27 outputs, to output unit 28, an estimation result indicating that the fatigue level of 7.9 is accumulated for the shoulders, the fatigue level of 7.7 is accumulated for the back, and the fatigue level of 5.7 is accumulated for the lower back.

When a plurality of data sets each including a calculated fatigue level and an obtained feeling of fatigue are thus accumulated, it is possible to obtain a correlation between a calculated fatigue level and an obtained feeling of fatigue. For example, FIG. 8 is a diagram for explaining fatigue level correction according to the embodiment. FIG. 8 illustrates a graph in which accumulated data sets are plotted with an X-axis indicating an obtained feeling of fatigue and a Y-axis indicating a calculated fatigue level. In the example in FIG. 8, when obtaining a correlated function by regression analysis (see the dashed line in FIG. 8), for example, the gradient of the function is 1 or less since each of the calculated fatigue levels indicates a higher value than a corresponding one of the obtained fatigue levels.

Accordingly, by substituting a calculated fatigue level into the correlated function described above, it is possible, without receiving any input from subject 11, to estimate a fatigue level that gives a less feeling of strangeness to subject 11.

An image is output from output unit 28 to display device 105. The result of the output will be described with reference to FIG. 9 and FIG. 10. FIG. 9 is a first diagram illustrating information to be output from fatigue estimation system 200 according to the embodiment. FIG. 10 is a second diagram illustrating information to be output from fatigue estimation system 200 according to the embodiment.

As illustrated in FIG. 9, after output from output unit 28, image data indicating the fatigue level of subject 11 is displayed on display module 105a of display device 105. A display included in computer 100a of subject 11 is used for display device 105, but a different display may be used instead. For example, display device 105 may be a dedicated display for use in fatigue estimation system 200.

As illustrated in FIG. 9, the fatigue level of subject 11 is shown separately for each of body parts. Specifically, the image data separately shows “degree of stiff shoulders” indicating the fatigue level of the shoulders of subject 11, “degree of backache” indicating the fatigue level of the back of subject 11, and “degree of lower backache” indicating the fatigue level of the lower back of subject 11. In addition, the image data shows, as additional information, the location of each of the body parts for which the fatigue level is indicated in the figure of a person, comprehensive fatigue level assessment, comment and advice on the result of the fatigue level estimation, etc.

As illustrated in FIG. 10, some advice may be made on a posture that is relatively liable to accumulate fatigue. In the example in FIG. 10, for example, attention is given to fatigue posture A whose second fatigue level is the highest, and the image of fatigue posture A is displayed together with sentences describing that fatigue posture A is a posture that is specifically liable to accumulate fatigue.

### [Operation]

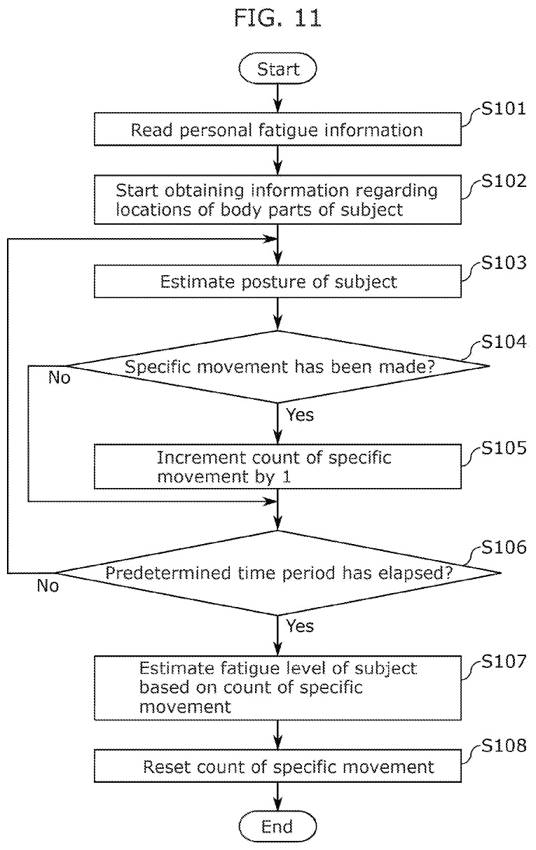

Next, an operation of fatigue estimation system 200 described above will be described with reference to FIG. 11. FIG. 11 is a flowchart illustrating an operation of fatigue estimation system 200 according to the embodiment.

In fatigue estimation system 200 according to the present embodiment, first, fatigue estimator 27 reads personal fatigue information stored in storage 24 (step S101). The personal fatigue information read herein is information in which a specific movement, a fatigue part, and information related to a first fatigue level are associated with one another.

Imaging device 101 starts operating in advance and images composing a video are sequentially output from imaging device 101. First obtainer 21 starts obtaining the images output (obtaining in step S102) and continues sequentially obtaining the images until fatigue estimation system 200 is stopped.

Posture estimator 25 estimates the posture of subject 11 based on the obtained images (step S103). Determiner 26 determines whether the movement of subject 11 due to a change in the posture of subject 11 estimated by posture estimator 25 matches a specific movement included in the personal fatigue information, to determine whether the specific movement has been made by subject 11 (step S104). When a plurality of specific movements are included in the personal fatigue information, the determination is sequentially performed for each of the specific movements.

When it is determined that subject 11 has made the specific movement (Yes in step S104), determiner 26 counts the specific movement by incrementing the count of the specific movement by 1 (step S105). After that, the process of the flowchart proceeds to step S106. When it is determined that subject 11 has not made the specific movement (No in step S104), the process skips step S105 and proceeds to step S106.

In step S106, determiner 26 determines whether a predetermined time period has elapsed. When it is determined that the predetermined time period has not elapsed (No in step S106), the process returns to step S103 and repeats the estimation of the posture of subject 11 and the determination on the presence or absence of a specific movement. When it is determined that the predetermined time period has elapsed (Yes in step S106), fatigue estimator 27 estimates the fatigue level of subject 11 based on the count of the specific movement (estimating in step S107). After that, estimation device 100 resets the count of the specific movement and ends the operation in preparation for the next fatigue level estimation.

In the present embodiment, it is thus possible to estimate the fatigue level of subject 11 based only on the determination of whether a specific movement has been made, as described above. In addition, it is also possible to combine the fatigue level estimation with a plurality of correction means for enhancement in the accuracy of the fatigue level of subject 11 to be estimated as well as for application to individual subject 11. It is thus possible to readily establish fatigue estimation system 200 providing accuracy demanded by, for instance, a manager who is in the position of managing subject 11 or the fatigue level of subject 11. In this way, fatigue estimation system 200 according to the present embodiment can estimate the fatigue level of subject 11 more appropriately.

### Advantageous Effects, etc.

As described above, fatigue estimation system 200 according to the present embodiment includes: an information output device (e.g., imaging device 101) that outputs information regarding the locations of body parts of subject 11; and estimation device 100 that, based on the information output from the information output device in a predetermined time period, estimates a fatigue level of subject 11 accumulated in the predetermined time period by counting a specific movement that appears in response to fatigue accumulation, and outputs the estimated fatigue level.

In such fatigue estimation system 200, based on whether a change in the estimated posture of subject 11 matches a specific movement that appears in response to fatigue accumulation, the specific movement made by subject 11 is counted. Since there is a correlation between the accumulation of the fatigue level of subject 11 and the count of the specific movement, it is possible to estimate the fatigue level of subject 11 by merely counting such a specific movement. Accordingly, computing for estimating the fatigue level of subject 11 is simplified. With fatigue estimation system 200, it is possible to estimate the fatigue level of subject 11 more appropriately.

For example, fatigue estimation system 200 may further include receiving device 102 that receives the input of a feeling of fatigue accumulated in the predetermined time period, where the feeling of fatigue is based on the subjective view of subject 11 and corresponds to the fatigue level of subject 11. Estimation device 100 may correct the fatigue level of subject 11 based on the feeling of fatigue and output the corrected fatigue level.

With the above features, it is possible to reflect a feeling of fatigue that is based on the subjective view of subject 11 in the estimated value of the fatigue level, thereby achieving fatigue level estimation which gives subject 11 a less feeling of strangeness. Accordingly, fatigue estimation system 200 according to the present embodiment can estimate the fatigue level of subject 11 more appropriately.

For example, fatigue estimation system 200 may further include a storage device (e.g., storage 24) that stores personal fatigue information of subject 11, where the personal fatigue information includes information related to a first fatigue level, and the first fatigue level is accumulated every time the specific movement is counted. Estimation device 100 may estimate the fatigue level of subject 11 by accumulating the first fatigue level based on the count of the specific movement.

With the above features, it is possible to perform fatigue level estimation that is more adapted to subject 11 based on the habit of each subject 11 which appears in the count of a specific movement. Accordingly, it is possible to achieve fatigue level estimation that gives subject 11 a less feeling of strangeness. Fatigue estimation system 200 can therefore estimate the fatigue level of subject 11 more appropriately.

For example, the personal fatigue information may include fatigue part information in which a fatigue part is associated with the specific movement, where the fatigue part is a body part of subject 11 for which the first fatigue level is accumulated every time the specific movement is counted.

With the above feature, it is possible to estimate the fatigue level of subject 11 for each of the body parts of subject 11. Accordingly, it is possible to estimate the fatigue level of subject 11 more appropriately since more detailed fatigue level estimation for each of the body parts can be achieved.

For example, when a posture of subject 11 estimated in a time period in the predetermined time period based on the information is defined as a fatigue posture, where the time period is from a predetermined timing until the specific movement of subject 11 is counted, estimation device 100 may (i) divide the first fatigue level by duration of the fatigue posture to calculate a second fatigue level, where the second fatigue level is the first fatigue level accumulated per unit time due to the fatigue posture, and (ii) correct the fatigue level of subject 11 using the second fatigue level calculated, and output the corrected fatigue level.

With the above feature, it is possible to estimate the fatigue level of subject 11 more accurately by using a second fatigue level that is based on a posture in a time period from a predetermined timing until a specific movement is made. Accordingly, it is possible to achieve more accurate fatigue level estimation, thereby estimating the fatigue level of subject 11 more appropriately.

For example, estimation device 100 may: estimate the posture of subject 11 based on the information output after the specific movement is last counted; determine whether the estimated posture of subject 11 matches the fatigue posture; and add a calculated value to the fatigue level of subject 11 and output a resultant value, where the calculated value is obtained by the second fatigue level being accumulated in accordance with duration of the posture that matches the fatigue posture.

With the above features, it is possible to perform fatigue level estimation based on a second fatigue level in a predetermined time period including a time period in which fatigue level estimation based on the count of a specific movement cannot be performed. Accordingly, it is possible to achieve more accurate fatigue level estimation, thereby estimating the fatigue level of subject 11 more appropriately.

For example, estimation device 100 may output presentation information for presenting the fatigue posture to subject 11.

With the above feature, it is possible to present, to subject 11, a fatigue posture that is relatively liable to accumulate fatigue, thereby contributing to subject 11's understanding of a posture that is liable to accumulate fatigue.

For example, fatigue estimation system 200 may further include obtaining device 103 that obtains personal information of subject 11 including at least one of age, sex, height, weight, a muscle mass, a stress level, a proportion of fat in a body, or proficiency in performing exercise. Estimation device 100 may correct the fatigue level of subject 11 using the personal information obtained, and output the corrected fatigue level.

With the above features, it is possible to perform more accurate fatigue level estimation through corrections based on various items of personal information. Accordingly, it is possible to estimate the fatigue level of subject 11 more appropriately.

For example, obtaining device 103 may obtain the personal information by connecting to external device 104 that stores a medical examination result including the personal information.

With the above feature, it is possible to obtain personal information all at once based on a medical examination result with which many items of personal information are managed all together. Accordingly, it is possible to readily implement more accurate fatigue level estimation through corrections based on various items of personal information, thereby estimating the fatigue level of subject 11 more appropriately.

For example, in a blank period which is a period in the predetermined time period and in which the information output device is unable to output the information, estimation device 100 may correct the fatigue level of subject 11 using a calculated value, and output the corrected fatigue level, where the calculated value is obtained by a preset supplementary fatigue level being accumulated in accordance with the length of the blank period.

According to the above feature, even in the case where subject 11 is not included in any of images and fatigue level estimation cannot be carried out, it is possible to perform supplementation using a supplementary fatigue level determined in advance, thereby enabling more accurate estimation of a fatigue level accumulated in a predetermined time period. Accordingly, it is possible to estimate the fatigue level of subject 11 more appropriately.

A fatigue estimation method according to the present embodiment includes: step S102 of obtaining information regarding locations of body parts of subject 11; and step S107 of, based on the information obtained, estimating the fatigue level of subject 11 accumulated in a predetermined time period by counting a specific movement that appears in response to fatigue accumulation in the predetermined time period.

With the fatigue estimation method described above, it is possible to obtain the same advantageous effects as those obtained by fatigue estimation system 200 described above.

Moreover, it is possible to implement the present embodiment as a non-transitory computer-readable recording medium having recorded thereon a program for causing a computer to execute the fatigue estimation method described above.

Accordingly, it is possible, with the use of a computer, to obtain the same advantageous effects as those obtained by the fatigue estimation method described above.

### Other Embodiments

Although an embodiment of the present disclosure is described above, the present disclosure is not limited to the embodiment.

For example, in the above embodiment, a process executed by a specific processing unit may be executed by another processing unit. An order of processes may be changed or processes may be executed in parallel.

The fatigue estimation system according to the present disclosure may be implemented by a plurality of devices each having one or more of the components of the fatigue estimation system or by a single device having all of the components. Likewise, the estimation device according to the present disclosure may be implemented by a plurality of devices each having one or more of the components of the estimation device or by a single device having all of the components. One or more of the functions of a component may be implemented as one or more functions of another component, or each of the functions may be distributed to any of components in any way. Any form with a configuration substantially including all of the functions achievable by the fatigue estimation system or the estimation device according to the present disclosure is included in the scope of the present disclosure.

In the above embodiment, the respective components may be implemented by executing software programs suited to the respective components. The respective components may be implemented by a program execution unit such as a CPU or a processor reading and executing a software program recorded on a recording medium such as a hard disk or semiconductor memory.

The respective components may be implemented by hardware. For example, the respective components may be circuits (or integrated circuits). These circuits may compose a single circuit as a whole or may be separate circuits. These circuits may be general-purpose or dedicated circuits.

General or specific aspects of the present disclosure may be implemented using a system, a device, a method, an integrated circuit, a computer program, or a computer-readable recording medium such as a CD-ROM, or any combination of systems, devices, methods, integrated circuits, computer programs, and recording media.

It is also possible to implement, as a method for estimating the posture of a subject, the present disclosure by a configuration that uses a location sensor besides a configuration that uses an imaging device. Specifically, the posture of a subject is estimated using a sensor module including a location sensor and a voltage sensor. Although it is described herein that a plurality of sensor modules are worn by a subject, the number of sensor modules to be worn by a subject is not particularly limited. Only one sensor module may be worn by a subject.

How to wear sensor modules is not particularly limited and any way may be allowed as long as the location of a predetermined body part of a subject can be measured. For example, a plurality of sensor modules are worn by a subject wearing clothing to which the plurality of sensor modules are attached.

A sensor module is a device that is worn by a subject on a predetermined body part and outputs information indicating a detection or measurement result in a manner linked to the predetermined body part. Specifically, the sensor module includes: a location sensor that outputs location information regarding the spatial location of the predetermined body part of the subject; and a voltage sensor that outputs potential information indicating an electric potential at the predetermined body part of the subject. Although a sensor module including both a location sensor and a voltage sensor is exemplified here, a voltage sensor is not essential if a sensor module includes a location sensor. A location sensor in such a sensor module is one example of an information output device that outputs information regarding the locations of the body parts of a subject. Accordingly, the information to be output is location information and includes the relative or absolute location of a predetermined body part of the subject. The information to be output may include, for example, potential information. The potential information is information including the value of an electric potential measured at a predetermined body part of the subject. Hereinafter, the location information and the potential information will be described in detail together with a location sensor and a voltage sensor.

A location sensor is a detector that detects the relative or absolute spatial location of a predetermined body part of a subject on which a sensor module is worn, and outputs information regarding the spatial location of the predetermined body part as the detection result. The information regarding the spatial location includes: information that can identify the location of a body part in a space, as described above; and information that can identify a change in the location of the body part resulting from body movement. Specifically, the information regarding the spatial location includes the locations of joints and bones in a space and information indicating changes in the locations.

A location sensor is composed by combining various sensors such as an acceleration sensor, an angular velocity sensor, a geomagnetic sensor, and a ranging sensor. Since location information output by the location sensor can be approximated to the spatial location of a predetermined body part of a subject, it is possible to estimate the posture of the subject from the spatial location of the predetermined body part.

A voltage sensor is a detector that measures an electric potential at a predetermined body part of a subject on which a sensor module is worn, and that outputs potential information indicating the electric potential at the predetermined body part as the measurement result. The voltage sensor is measuring equipment that includes electrodes and measures a potential generated between the electrodes using an electrometer. The potential information output by the voltage sensor indicates a potential generated at the predetermined body part of the subject. Since the potential corresponds to, for instance, the active potential of muscles in the predetermined body part, it is possible to enhance estimation accuracy in estimating the posture of the subject from, for instance, the active potential of the predetermined body part.

The fatigue estimation system according to one aspect of the present disclosure described herein estimates the fatigue level of a subject using the posture of the subject estimated as described above. Since the processes following the estimation of the posture of the subject are the same as those described in the above embodiment, description thereof is omitted.

Apart from the method for estimating the fatigue level of a subject described in the above embodiment, there is a method for estimating the fatigue level of a subject from the posture of the subject based on an expression that is a×muscle loading+b×joint loading+c×blood flow rate, where a, b, and c are coefficients (weighting coefficients if stated differently). The muscle loading and the joint loading are index amounts with no unit of quantity required, where an amount with a Newton unit is normalized within a range from 0 to 1 when a preset largest value is 1. The blood flow rate is an index amount with no unit of quantity required, within the range from 0 to 1 obtained as a ratio of the measured value of at least a default value to a default value. By defining the relationship of the coefficients in the above expression as a+b+c=1, a fatigue level calculated using the above expression is also calculated as a value within the range from 0 to 1.

The above expression is an example of using three index amounts, but the estimation of the fatigue level of a subject can be performed if at least one of the three index amounts is used. In this case, by defining the sum of the weighting coefficients, each of which is multiplied by a different one of the index amounts, to be 1, the fatigue level of the subject is calculated as a value within the range from 0 to 1, as is the case above.

With such other method, however, it is difficult to perform fatigue level estimation adapted to each person such as the one described in the foregoing embodiment. In addition, it is difficult to adjust various parameters so that a fatigue level is estimated for each person. In view of this, a fatigue level, which is accumulated in a time period before a specific movement is counted and calculated in the foregoing embodiment, is calculated a plurality of times using another method. Since each of the results of the calculations performed the plurality of times is equivalent to the fatigue level accumulated in the time period, the same fatigue level can be obtained. In other words, by adjusting various parameters so that the results of the calculations performed the plurality of times are all same, parameters with which a fatigue level appropriate for each person is estimated even with another method are determined.

As described above, as another method for estimating the fatigue level of a subject, the estimation device can (i) estimate the fatigue level of the subject based on an expression that is a×muscle loading+b×joint loading+c×blood flow rate, where a, b, and c are coefficients, (ii) calculate a fatigue level in a time period a plurality of times based on the expression, where the time period is a period before a specific movement is counted, and (iii) correct a, b, and c based on the result of the calculation performed the plurality of times.

With the above features, it is possible to correct the coefficients a, b, and c for estimation results based on the expression that is a×muscle loading+b×joint loading+c×blood flow rate which is used as the other method, so that a fatigue level appropriate for each person is estimated, which is described in the foregoing embodiment. In other words, it is possible to correct other fatigue level estimation method for adaptation to each person based on fatigue levels calculated in the present embodiment, and also utilize the fatigue levels for expanding the general versatility of the other fatigue level estimation method.

The present disclosure may be implemented as a fatigue estimation system or a fatigue estimation method to be executed by an estimation device. The present disclosure may be implemented also as a program for causing a computer to execute such a fatigue estimation method, or as a non-transitory computer-readable recording medium having such a program recorded thereon.

Various modifications to the embodiments which may be conceived by those skilled in the art, as well as embodiments resulting from arbitrary combinations of elements and functions from different embodiments are included within the scope of the present disclosure so long as they do not depart from the essence of the present disclosure.

### REFERENCE SIGNS LIST

11 subject
24 storage (storage device)
100 estimation device
101 imaging device (information output device)
102 receiving device
103 obtaining device
104 external device
200 fatigue estimation system

1. A fatigue estimation system comprising:
an information output device that outputs information regarding locations of body parts of a subject; and
an estimation device that, based on the information output from the information output device in a predetermined time period, estimates a fatigue level of the subject accumulated in the predetermined time period by counting a specific movement that appears in response to fatigue accumulation, and outputs the estimated fatigue level.

2. The fatigue estimation system according to claim 1, further comprising:
a receiving device that receives an input of a feeling of fatigue accumulated in the predetermined time period, the feeling of fatigue being based on a subjective view of the subject and corresponding to the fatigue level of the subject, wherein
the estimation device corrects the fatigue level of the subject based on the feeling of fatigue and outputs the corrected fatigue level.

3. The fatigue estimation system according to claim 1, further comprising:
a storage device that stores personal fatigue information of the subject, the personal fatigue information including information related to a first fatigue level, the first fatigue level being accumulated every time the specific movement is counted, wherein
the estimation device estimates the fatigue level of the subject by accumulating the first fatigue level based on the count of the specific movement.

4. The fatigue estimation system according to claim 3, wherein
the personal fatigue information includes fatigue part information in which a fatigue part is associated with the specific movement, the fatigue part being a body part of the subject for which the first fatigue level is accumulated every time the specific movement is counted.

5. The fatigue estimation system according to claim 3, wherein
when a posture of the subject estimated in a time period in the predetermined time period based on the information is defined as a fatigue posture, the time period being from a predetermined timing until the specific movement of the subject is counted, the estimation device (i) divides the first fatigue level by duration of the fatigue posture to calculate a second fatigue level, the second fatigue level being the first fatigue level accumulated per unit time due to the fatigue posture, and (ii) corrects the fatigue level of the subject using the second fatigue level calculated, and outputs the corrected fatigue level.

6. The fatigue estimation system according to claim 5, wherein
the estimation device:
estimates a posture of the subject based on the information output after the specific movement is last counted;
determines whether the estimated posture of the subject matches the fatigue posture; and
adds a calculated value to the fatigue level of the subject and outputs a resultant value, the calculated value being obtained by the second fatigue level being accumulated in accordance with duration of the posture that matches the fatigue posture.

7. The fatigue estimation system according to claim 5, wherein
the estimation device outputs presentation information for presenting the fatigue posture to the subject.

8. The fatigue estimation system according to claim 1, further comprising:
an obtaining device that obtains personal information of the subject including at least one of age, sex, height, weight, a muscle mass, a stress level, a proportion of fat in a body, or proficiency in performing exercise, wherein
the estimation device corrects the fatigue level of the subject using the personal information obtained, and outputs the corrected fatigue level.

9. The fatigue estimation system according to claim 8, wherein
the obtaining device obtains the personal information by connecting to an external device that stores a medical examination result including the personal information.

10. The fatigue estimation system according to claim 1, wherein
in a blank period which is a period in the predetermined time period and in which the information output device is unable to output the information, the estimation device corrects the fatigue level of the subject using a calculated value, and outputs the corrected fatigue level, the calculated value being obtained by a preset supplementary fatigue level being accumulated in accordance with a length of the blank period.

11. The fatigue estimation system according to claim 1, wherein
the estimation device:
as another method for estimating the fatigue level of the subject, estimates the fatigue level of the subject based on a relational expression that uses (i) at least one of index amounts that are muscle loading, joint loading, and a blood flow rate, and (ii) weighting coefficients each of which is multiplied by a different one of the index amounts; and
corrects the weighting coefficients based on a result of calculating a fatigue level in a time period a plurality of times, the time period being a period before the specific movement is counted.

12. The fatigue estimation system according to claim 1, wherein
the estimation device:
as another method for estimating the fatigue level of the subject, estimates the fatigue level of the subject based on an expression that is a×muscle loading+b×joint loading+c×blood flow rate, where a, b, and c are coefficients;
calculates a fatigue level in a time period a plurality of times based on the expression, the time period being a period before the specific movement is counted; and
corrects a, b, and c based on a result of the calculation performed the plurality of times.

13. A fatigue estimation method comprising:
obtaining information regarding locations of body parts of a subject; and
based on the information obtained, estimating a fatigue level of the subject accumulated in a predetermined time period by counting a specific movement that appears in response to fatigue accumulation in the predetermined time period.

14. A non-transitory computer-readable recording medium having recorded thereon a program for causing a computer to execute the fatigue estimation method according to claim 13.
